(19) United States
(12) Patent Application Publication
(10) Pub. No.: US 2011/0015543 A1
(43) Pub. Date: Jan. 20, 2011

(54) FLUID SAMPLE COLLECTION SYSTEM

(76) Inventors: NATHANIEL G. BUTLIN, (US); Charles A. Martin, (US)

Correspondence Address: MEHLIN DEAN MATTHEWS, P.O. BOX 3340, ALBANY, OR, 97321, US

(21) Appl. No.: 12/838,823

(22) Filed: Jul. 19, 2010

Related U.S. Application Data
(60) Provisional application No. 61/271,110, filed on Jul. 17, 2009.
(60) Provisional application No. 61/283,284, filed on Dec. 2, 2009.

(51) Int. Cl. A61B 5/00

(52) U.S. Cl. 600/573

(57) ABSTRACT

A sample collection container adapted to be connected to, and disconnected from, a sample collection adapter and a sample collection container cap. The sample collection adapter has a downward sloping interior surface that provides a free-flowing path for samples provided by a subject to the sample collection container. The sample collection adapter may have a three-dimensional rim surface that is curved in two different non-parallel planes.

### CROSS-REFERENCE TO RELATED APPLICATIONS

This application claims priority of. U.S. Provisional Application No. 61/271,110, filed Jul. 17, 2009. This application is also related to U.S. Provisional Application No. 61/283,284, filed Dec. 2, 2009. The two aforementioned applications are herein expressly incorporated by reference in their entirety.

### BACKGROUND OF THE INVENTION

1. Field of the Invention

The present invention relates to fluid sample collection. In particular, the invention relates to a system for collecting oral fluid samples.

2. Description of Related Art

Small quantities of materials for analysis are frequently required from subjects undergoing testing. For example, oral fluids may be required. One approach to oral fluid sample collection is to place an absorbent material in the oral cavity so that a quantity may be absorbed and subsequently transferred to a container along the with absorbent material.

Another common technique for sample collection is to have a subject spit into a container. Such containers may have an attachment to aid in sample collection and serve as a source for additional fluid to be mixed with the sample being obtained. The additional fluid may serve to wash the sample into the container.

For oral fluid samples that are collected without the use of absorbents or the addition of other fluid, the efficiency of sample collection is dependent upon the geometry of the collection device, and it is desirable that the device used for collection provide a free-flowing path to the sample container.

A subject providing an oral fluid sample may be less than comfortable with the act of expectorating into a container, and it is desirable that a collection device be easy to use and minimize discomfort on the part of the subject.

Thus, a need exists for an efficient system for collecting oral fluid samples without absorbents or admixtures. There is also a need for a sample collection system that is easy to use.

### BRIEF SUMMARY OF THE INVENTION

The present invention provides a sample collection adapter that couples to a sample collection container. The adapter funnels a liquid sample provided by a subject into a container. The adapter may subsequently be replaced by a cap to seal the sample in the sample collection container.

In one embodiment of the invention the sample collection adapter is a conical funnel. The conical funnel has a thread that allows the funnel to be connected to and disconnected from a sample collection container with a mated thread.

In another embodiment, the sample collection adapter is ergonomically shaped to assist in the collection of liquid samples. The rim of the proximal opening of the sample collection adapter is a non-planar three-dimensional surface with portions that are curved in at least two different planes.

### BRIEF DESCRIPTION OF THE DRAWINGS

FIG. 1A shows a side view of a sample collection system in accordance with an embodiment of the present invention.

FIG. 1B shows a section view of the sample collection system of FIG. 1A.

FIG. 2 shows a top view of the funnel device shown in FIG. 1A.

FIG. 3 shows a side view of an assembled sample collection device in accordance with an embodiment of the present invention.

FIG. 4A shows a side view of an ergonomic sample collection system in accordance with an embodiment of the present invention.

FIG. 4B shows a section view of an ergonomic sample collection system shown in FIG. 4A.

FIG. 5A shows an orthogonal side view of the ergonomic funnel device shown in FIG. 4A.

FIG. 5B shows a top view of the ergonomic funnel device shown in FIG. 5.

FIG. 6 shows a side view of an assembled ergonomic sample collection device in accordance with an embodiment of the present invention.

### DETAILED DESCRIPTION OF THE INVENTION

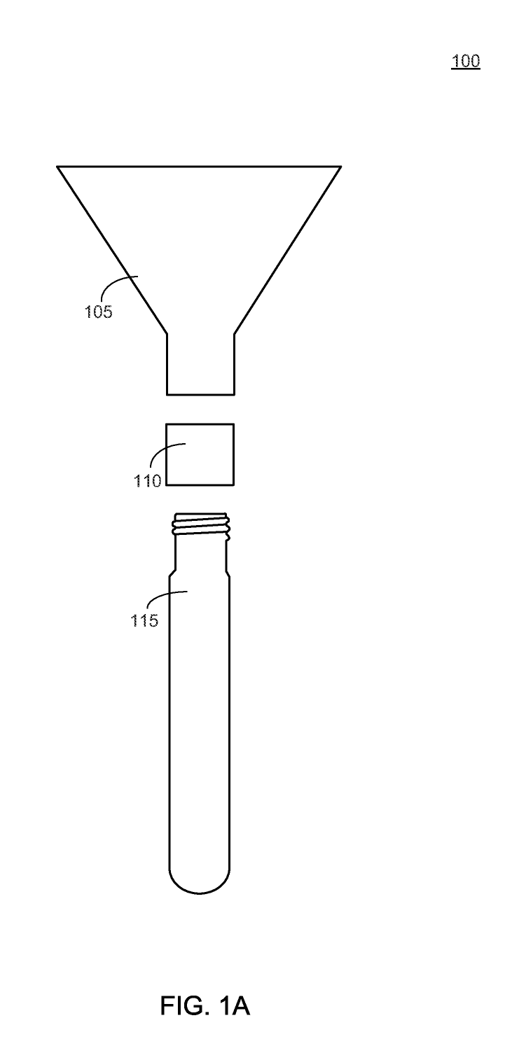

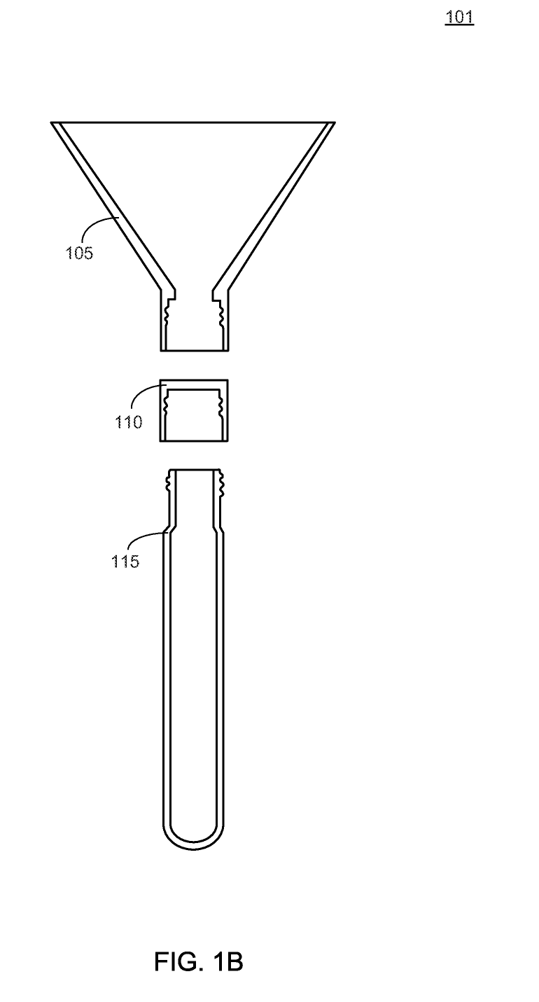

FIG. 1A shows a side view 100 of an embodiment of a sample collection system. The sample collection system includes a conical funnel 105, a container cap 110 and a sample container 115. FIG. 1B shows a section view 101 of the sample collection system of FIG. 1A. The conical funnel 105 has a female thread that allows it to be connected to and disconnected from the sample container 115 for collection of a sample. The container cap 110 has a female thread that allows it to be connected to and sample container 115 after collection of a sample in order to provide a seal for the sample container 115.

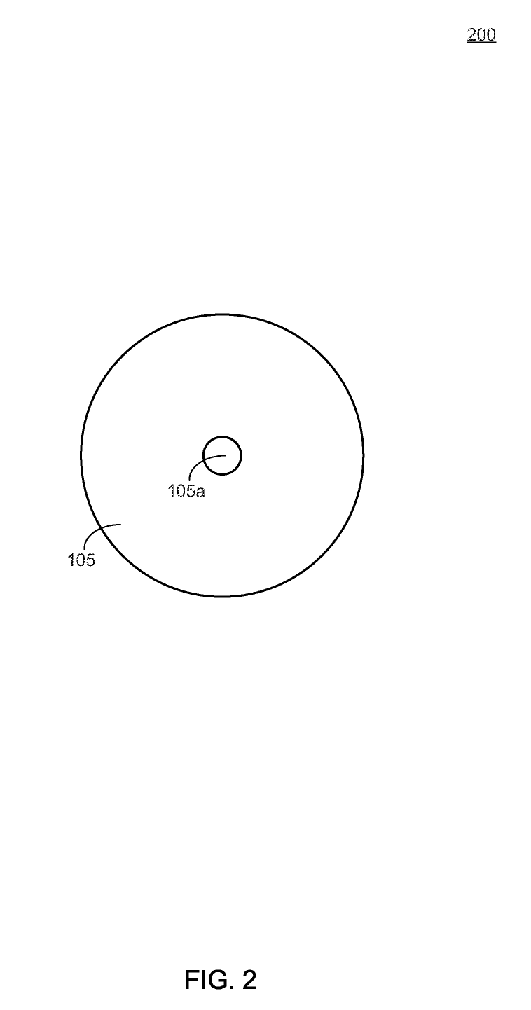

The conical funnel 105 has a downward sloping inner surface that provides a free-flowing path from the upper opening to the lower opening. FIG. 2 shows a top view of the funnel device shown in FIG. 1A. In one embodiment the diameter of the aperture 105a is equivalent to the inner diameter of the opening of the sample container 115.

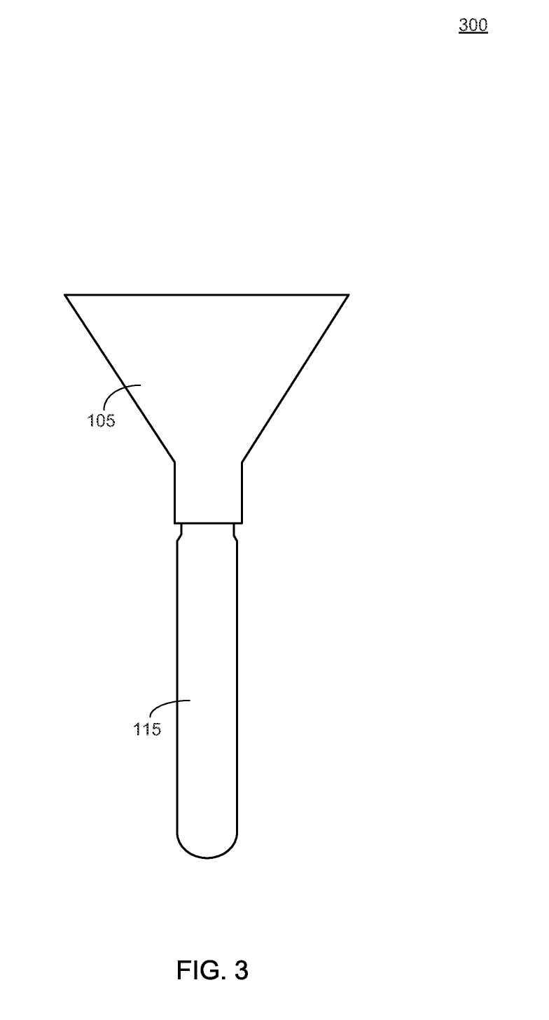

FIG. 3 shows a side view 300 of an embodiment of an assembled sample collection device, with the conical funnel 105 connected to the sample container 115. The conical funnel 105 may be replaced by the container cap 110 of FIG. 1A after a sample has been collected. The conical funnel 105 and container cap 110 may provide a liquid-tight seal when connected to said sample container 115.

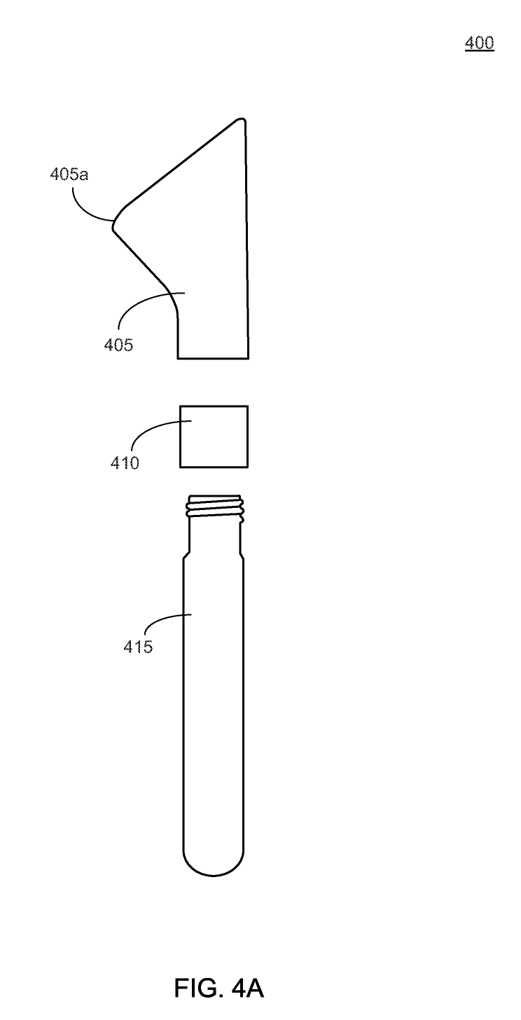

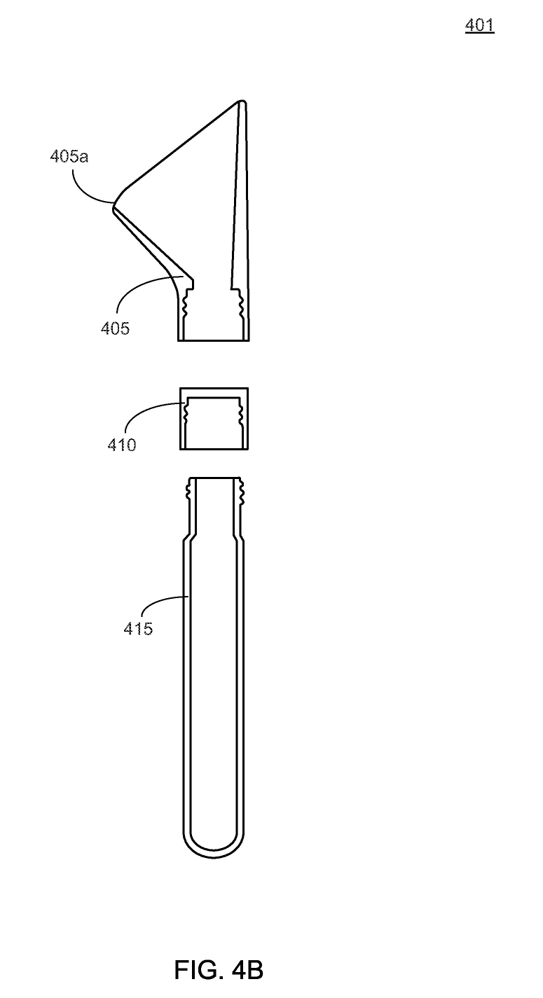

FIG. 4A shows a side view 400 of an embodiment of a sample collection system. The sample collection system includes an ergonomic funnel 405, a container cap 410 and a sample container 415. FIG. 4B shows a section view 401 of the ergonomic sample collection system shown in FIG. 4A. The rim 405a of the ergonomic funnel 405 is a three-dimensional surface that resides largely in a first plane, as can be seen in FIGS. 4A and 4B, and has a lower portion that is concave in a second non-parallel plane. The three-dimensional surface provides enhanced conformation to the subject providing a sample.

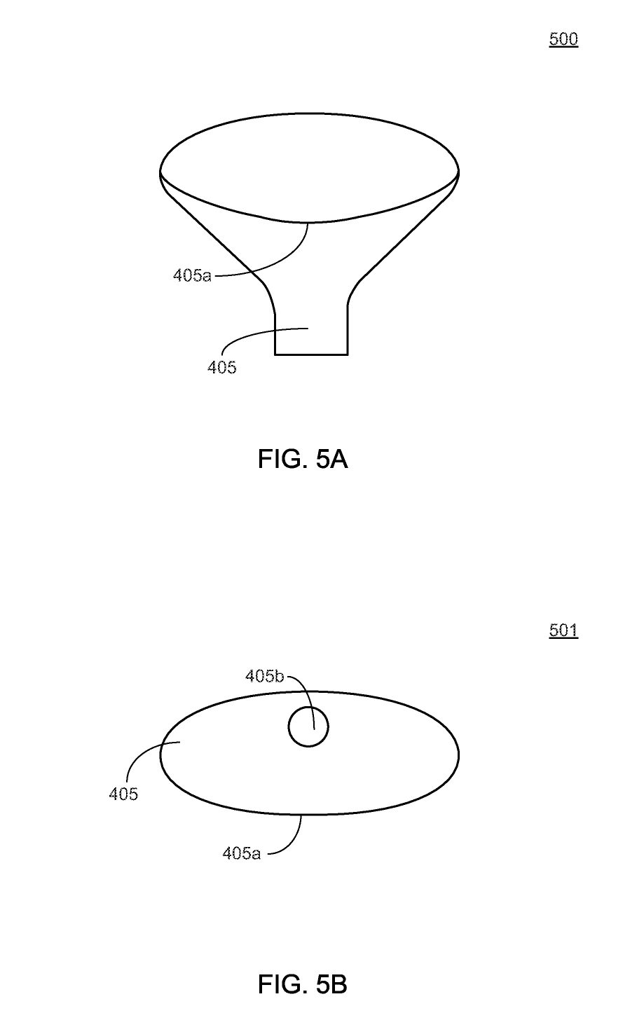

FIG. 5A shows an orthogonal side view 500 of the ergonomic funnel device shown in FIGS. 4A and 4B. FIG. 5B shows a top view 501 of the ergonomic funnel device shown in FIG. 5. The ergonomic funnel 405 has a downward sloping inner surface that provides a free-flowing path from the upper opening to the lower opening. In one embodiment the diameter of the aperture 405b is equivalent to the inner diameter of the opening of the sample container 115. The projection of the rim 405a along the axis of the aperture 405b may be an asymmetric oval.

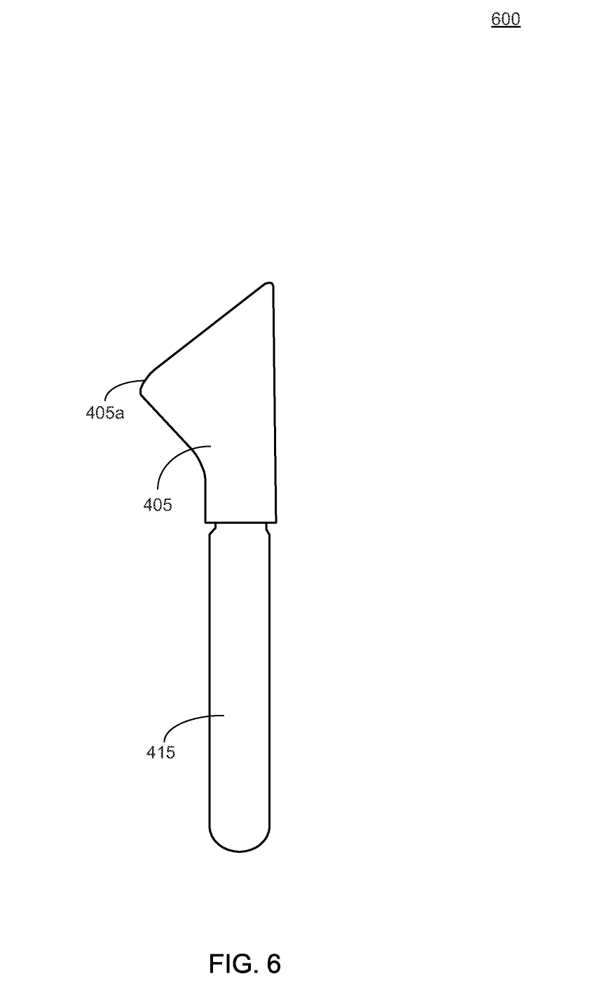

FIG. 6 shows a side view 600 of an embodiment of an assembled ergonomic sample collection device, with the ergonomic funnel 405 connected to the sample container 415. The ergonomic funnel 405 may be replaced by the container cap 410 of FIG. 4A after a sample has been collected. The ergonomic funnel 405 and container cap 410 may provide a liquid tight seal when connected to said sample container 415.

While the invention has been described in detail with reference to preferred embodiments thereof, it will be apparent to one skilled in the art that various changes can be made, and equivalents employed, without departing from the scope of the invention.

What is claimed:

1. A sample collection system comprising:
a sample collection container;
a sample collection adapter for receiving a sample, wherein said sample collection adapter comprises a downward sloping interior surface and is adapted to be connected to, and disconnected from, said sample collection container; and
a sample collection container cap, wherein said sample collection container cap is adapted to be connected to and disconnected from said sample collection container.

2. The sample collection system of claim 1 wherein said sample collection adapter is a conical funnel.

3. The sample collection system of claim 1 wherein said sample collection adapter is an ergonomic funnel.

4. The sample collection system of claim 1 wherein said sample collection adapter comprises a rim having a curvature in a first plane and a concave curvature in a second plane.

5. The sample collection system of claim 1 wherein said sample collection adapter, comprises a female thread.

6. The sample collection system of claim 1 wherein said sample container comprises a male thread.

7. The sample collection system of claim 1 wherein said sample collection container cap comprises a female thread.

8. The sample collection system of claim 1 wherein said sample collection adapter comprises an aperture that has a diameter equivalent to an inner diameter of an opening in said sample collection container.

9. A sample collection system comprising:
a sample collection container;
a sample collection adapter for receiving a sample, wherein said sample collection adapter comprises a conical interior surface and is adapted to be connected to, and disconnected from, said sample collection container; and
a sample collection container cap, wherein said sample collection container cap is adapted to be connected to and disconnected from said sample collection container.

10. The sample collection system of claim 9 wherein said sample collection adapter comprises a female thread.

11. The sample collection system of claim 9 wherein said sample container comprises a male thread.

12. The sample collection system of claim 9 wherein said sample collection container cap comprises a female thread.

13. The sample collection system of claim 9 wherein said sample collection adapter comprises an aperture that has a diameter equivalent to an inner diameter of an opening in said sample collection container.

14. A sample collection system comprising:
a sample collection container;
a sample collection adapter for receiving a sample, wherein said sample collection adapter comprises an ergonomic funnel and is adapted to be connected to, and disconnected from, said sample collection container; and
a sample collection container cap, wherein said sample collection container cap is adapted to be connected to and disconnected from said sample collection container.

15. The sample collection system of claim 14 wherein said sample collection adapter comprises a female thread.

16. The sample collection system of claim 14 wherein said sample container comprises a male thread.

17. The sample collection system of claim 14 wherein said sample collection container cap comprises a female thread.

18. The sample collection system of claim 14 wherein said sample collection adapter comprises an aperture that has a diameter equivalent to an inner diameter of an opening in said sample collection container.

19. The sample collection system of claim 14 wherein said ergonomic funnel comprises a rim having a curvature in a first plane and a concave curvature in a second plane.

20. The sample collection system of claim 19 wherein said ergonomic funnel comprises an aperture that has a diameter equivalent to an inner diameter of an opening in said sample collection container.
